(19) United States
(12) Patent Application Publication
(10) Pub. No.: US 2011/0117064 A1
(43) Pub. Date: May 19, 2011

(54) ASSAY FOR THE PREDICTION OF THERAPEUTIC EFFECTIVENESS OF MESENCHYMAL STROMAL CELLS, AND METHODS OF USING SAME

(76) Inventor: CHRISTOF WESTENFELDER, Salt Lake City, UT (US)

(21) Appl. No.: 12/903,935

(22) Filed: Oct. 13, 2010

Related U.S. Application Data
(60) Provisional application No. 61/251,168, filed on Oct. 13, 2009.

(51) Int. Cl. A61K 35/12; A61P 1/00; A61P 17/00; A61P 9/00; A61P 21/00; A61P 19/00; A61P 3/00; A61P 13/12; A61P 3/10; C12Q 1/02

(52) U.S. Cl. 424/93.7; 435/29

(57) ABSTRACT

The invention relates to assays for testing the therapeutic effectiveness of mesenchymal stromal cell (MSC) populations and methods of treating pathologies with passaged and/or frozen and thawed MSC populations.

### RELATED APPLICATIONS

This application claims priority from U.S. Provisional Application 61/251,168, filed on Oct. 13, 2009, which is incorporated herein, by reference, in its entirety.

### FIELD OF THE INVENTION

The present invention generally relates to assays that predict the therapeutic effectiveness of mesenchymal stromal cells.

### BACKGROUND OF THE INVENTION

Stem cell therapy offers a promising new option for the treatment of complex diseases. While there are ethical and tumorigenic concerns with embryonic stem cells, adult stem cells are already used successfully and safely to treat patients. Mesenchymal stromal cells (MSCs) are bone marrow derived adherent fibroblast-like cells that differentiate into a large number of cell types, have immunomodulatory properties and secrete cytokines and growth factors (Schinkothe T, et al., Stem Cells Dev. 2008; 17: 199-206), together making them potential ideal candidates for therapies of various disorders (Porada C D, et al., Curr Stem Cell Res Ther. 2006; 1:365-9). MSCs have been used successfully to treat a number of diseases in animal models and are currently used in clinical trials to treat different diseases including myocardial infarction, graft versus host disease, Crohn's disease and others (Giordano A, et al., J Cell Physiol. 2007; 211: 27-35). MSCs are effective in reducing renal injury and enhancing recovery of renal function in animal models of acute kidney injury (AKI), including an ischemia/reperfusion as well as a cisplatinum toxicity model, but do not or only rarely contribute to differentiated renal cell types, e.g. tubular cells or endothelial cells (Humphreys B D, et al. Minerva Urol Nefrol. 2006; 58: 329-37). Growth factors including IGF-1 (Imberti B, et al., J Am Soc Nephrol. 2007; 18: 2921-8), EGF and vasculotropic factors (Togel F, et al. Am J Physiol Renal Physiol. 2007; 292: F1626-35) have been shown to be mediators of renal repair and this effect can be reproduced using MSC conditioned medium (Bi B, Schmitt R, et al. J Am Soc Nephrol. 2007; 18: 2486-96).

In order to use MSCs effectively a sufficient number of cells is needed to form an adequate dose. Thus, in some situations, MSCs must be expanded to provide a sufficient number of cells for a therapeutic effective dose and/or frozen in order to provide a dose at a clinically relevant time. The effectiveness of MSCs in treating various pathologies must be confirmed when the cells are passaged, expanded or frozen.

The present invention provides assays that show when MSCs are still effective for use in treatment of various pathologies despite passaging, freezing and or expansion. The present invention also provides methods of using passaged and/or frozen MSCs for the treatment of pathologies are also provided.

### SUMMARY OF THE INVENTION

The invention provides a method of assaying the thereapeutic effectiveness of mesenchymal stromal cells (MSCs) for treating a pathology in a subject comprising: isolating a first population of MSCs, wherein the first population of MSCs has been freshly isolated; isolating a second population of MSCs, wherein the second population has been passaged and/or frozen and thawed; measuring the expression of stromal derived factor-1 (SDF-1) and/or vascular endothelial growth factor (VEGF) in the first and second populations; and comparing the expression of SDF-1 and/or VEGF in the first and second populations; wherein, if the expression of SDF-1 and/or VEGF in the second population is the same as or greater than the expression of SDF-1 and/or VEGF in the first population, the second population contains MSCs that are therapeutically effective.

In one embodiment of the method of assaying the thereapeutic effectiveness of mesenchymal stromal cells (MSCs) for treating a pathology in a subject, the MSCs from the first and second populations are autologous to the subject. Preferably, the subject is a mammal. More preferably, the mammal is a human.

In another embodiment of the method of assaying the thereapeutic effectiveness of mesenchymal stromal cells (MSCs) for treating a pathology in a subject, the MSCs from the first and second populations are allogeneic to the subject. Preferably, the subject is a mammal. More preferably, the mammal is a human.

In another embodiment of the method of assaying the thereapeutic effectiveness of mesenchymal stromal cells (MSCs) for treating a pathology in a subject, the MSCs from the first and second populations are isolated at different times. Optionally, the time between the isolation of the first and second populations is about 1 day, 1 week, 1 month, 1 year or greater than 1 year apart.

In another embodiment of the method of assaying the thereapeutic effectiveness of mesenchymal stromal cells (MSCs) for treating a pathology in a subject, the first and second populations are isolated at about the same time.

In another embodiment of the method of assaying the thereapeutic effectiveness of mesenchymal stromal cells (MSCs) for treating an MSC related pathology in a subject. In certain embodiments, the pathology is selected from the group consisting of a neurological pathology, an inflammatory pathology, a renal pathology, a hepatic pathology, a cardiovascular pathology, a retinal pathology, a muscular pathology, a bone-related pathology, a gastrointestinal pathology, a skin related pathology and a metabolic pathology. Optionally, the renal pathology is selected from the group consisting of acute kidney injury, acute renal failure, chronic renal failure, chronic kidney disease and transplant. Optionally, the neurological pathology is stroke. Optionally, the inflammatory pathology is multi-organ failure. Optionally, the metabolic pathology is diabetes.

The invention also provides a method of treating an MSC related pathology in a subject in need thereof comprising: isolating a first population of MSCs, wherein the first population of MSCs has been freshly isolated; isolating a second population of MSCs, wherein the second population has been passaged one or more times and/or frozen and thawed; measuring the expression and/or secretion into the media of stromal derived factor-1 (SDF-1) and/or vascular endothelial growth factor (VEGF) in the first and second populations; and comparing the expression of SDF-1 and/or VEGF in the first and second populations; wherein, if the expression of SDF-1 and/or VEGF in the second population is the same as or greater than the expression of SDF-1 and/or VEGF in the first population the second population contains MSCs that are therapeutically effective; and a therapeutically effective dose of the MSCs in the second population is administered to the subject, thereby treating the MSC related pathology in the subject.

In one embodiment of the method of treating an MSC related pathology in a subject in need thereof, the MSCs from the first and second populations are autologous to the subject. Preferably, the subject is a mammal. More preferably, the mammal is a human.

In another embodiment of the method of treating an MSC related pathology in a subject in need thereof, the MSCs from the first and second populations are allogeneic to the subject. Preferably, the subject is a mammal. More preferably, the mammal is a human.

In another embodiment of the method of treating an MSC related pathology with MSCs in a subject in need thereof, the MSCs from the first and second populations are isolated at different times. Optionally, the time between the isolation of the first and second populations is about 1 day, 1 week, 1 month, 1 year or greater than 1 year apart. In another embodiment of the method of treating an MSC related pathology in a subject in need thereof, the first and second populations are isolated at about the same time.

In another embodiment of the method of treating an MSC related pathology the pathology is selected from the group consisting of a neurological pathology, an inflammatory pathology, a renal pathology, a hepatic pathology, a cardiovascular pathology, a retinal pathology, a muscular pathology, a bone-related pathology, a gastrointestinal pathology, a skin-related pathology and a metabolic pathology.

Optionally, the renal pathology is selected from the group consisting of acute kidney injury, acute renal failure, chronic renal failure, chronic kidney disease and transplant. Optionally, the neurological pathology is stroke. Optionally, the inflammatory pathology is multi-organ failure. Optionally, the metabolic pathology is diabetes.

The invention also provides a kit comprising reagents for the detection of the expression of SDF-1 and reagents for the detection of VEGF. In one embodiment of the kit, the kit further comprising reagents for culturing MSCs. In another embodiment of the kit, the kit further comprising reagents for freezing MSCs. In another embodiment of the kit, the reagents for the detection of SDF-1 or VEGF comprise reagents for use in an enzyme linked immunosorbent assay (ELISA). In another embodiment of the kit, the detection of SDF-1 or VEGF comprise reagents for use with reverse transcriptase polymerase chain reaction (rtPCR).

The invention also provides a method of producing a dosage form of MSCs comprising: isolating a first population of MSCs, wherein the first population of MSCs has been freshly isolated; isolating a second population of MSCs, wherein the second population has been passaged one or more times and/or frozen and thawed; measuring the expression of stromal derived factor-1 (SDF-1) and/or vascular endothelial growth factor (VEGF) in the first and second populations; and comparing the expression of SDF-1 and/or VEGF in the first and second populations; wherein, if the expression of SDF-1 and/or VEGF in the second population is the same as or greater than the expression of SDF-1 and/or VEGF in the first population the second population of MSCs may be combined with a physiologically acceptable solution, thereby producing a dosage form of MSCs.

In one embodiment of the method of producing a dosage form of MSCs, the MSCs from the first and second populations are autologous to the subject. Preferably, the subject is a mammal. More preferably, the mammal is a human.

In another embodiment of the method of producing a dosage form of MSCs, the MSCs from the first and second populations are allogeneic to the subject. Preferably, the subject is a mammal. More preferably, the mammal is a human.

In another embodiment of the method of producing a dosage form of MSCs, the

MSCs from the first and second populations are isolated at different times. Optionally, the time between the isolation of the first and second populations is about 1 day, 1 week, 1 month, 1 year or greater than 1 year apart.

In another embodiment of the method of producing a dosage form of MSCs, the first and second populations are isolated at about the same time.

### BRIEF DESCRIPTION OF THE DRAWINGS

FIG. 1 is a schematic of a protocol for the formation of a dosage form of MSCs.

FIG. 2 contains two bar graphs showing VEGF gene regulation and protein expression in MSCs in response to siRNA. Left panel: Absolute gene regulation determined by real time quantitative RT-PCR. Right panel: VEGF protein concentrations determined by ELISA in tissue culture supernatant of cells treated with VEGF siRNA after 24 and 48 hrs after the end of the siRNA incubation period (24 hrs). VEGF knockdown on mRNA and protein level was highly significant (P<0.01, t-test).

FIG. 3 is a bar graph showing inhibition of the proliferation of NRK cells with MSCs in which VEGF is knocked down in an in vitro study to determine the effect of VEGF knockdown on proliferation of MSC-conditioned medium (MSC CM) on NRK cells using the MTT assay. Data are shown as mean+S.D. (n=6 per group). S.F.=serum free medium. (−) siRNA=negative control (irrelevant) siRNA. MSC CM after knockdown of VEGF significantly reduced proliferation of NRK cells compared to negative control siRNA (P=0.029), and control MSC CM (P=0.038). Addition of 10 ng/ml VEGF (S.F.+VEGF) brought proliferation back to baseline. 10% FBS as positive control showed the highest proliferative activity.

FIG. 4 shows results of an in vivo study to determine the effect of VEGF knockdown on therapeutic effectiveness in an ischemia/reperfusion model of AKI in rats. FIG. 4A (left panel) is a bar graph showing that VEGF knockdown MSCs administered to AKI induced rats results in higher serum creatinine than administration of MSCs with normal amounts of VEGF. Regular MSCs (grey bars) were renoprotective and enhance recovery from AKI in rats compared to VEGF knockdown MSCs (black bars).

FIG. 4B (right panel) is a line graph showing greater mortality in AKI induced rats administered VEGF knockdown MSCs when compared to control MSCs. Survival was increased in animals treated with wild type MSCs compared to VEGF knockdown MSCs (P<0.05; n=8).

FIG. 5 shows an assessment of micro-vessel density in renal cortex sections of rats 4 weeks after AKI.

FIG. 5A is a CD34 staining of renal vasculature without nuclear counterstaining.

FIG. 5B is a binary image of FIG. 5A made with ImageJ to determine the area of the stained vessels.

FIG. 5C is a bar graph showing a decrease in vascular area as a result of administration of VEGF knockdown MSCs to AKI induced rats when compared to controls MSCs. The bars represent the calculated mean vascular area (percent of section) per visual field in the renal cortex. Three 20× field per section from every group (n=5) were randomly chosen and averages plotted. Animals treated with normal MSCs have a significantly higher vascular area compared to VEGF knockdown MSC treated animals and controls (vehicle treated).

FIG. 6 is a bar graph showing SDF-1 protein expression in MSCs in response to siRNA. 1 ml medium from wells of cultured MSCs of equal cell density was analyzed by ELISA for SDF-1 protein on days 2, 3 and 4 post-transfection with siRNAs. Shown are SDF-1 concentrations [ng/ml] from three independent cultures for control (non-transfected) cells (black bar), cells treated with transfection agent alone (dark grey bar), cells transfected with nonsense RNA (light grey bar, (−) siRNA), and cells transfected with SDF-1 siRNA (blue bar).

FIG. 7A is a bar graph showing that SDF-1 knockdown MSCs administered to AKI induced rats results in higher serum creatinine (SCr) than administration of MSCs with normal SDF-1 levels. The bars represent SCr levels [mg/dL] in rats treated with vehicle alone (yellow bars), normal MSCs (blue bars), and SDF-1 knock-down MSCs (green bars) prior to induction of AKI (BL), at day 1 (D1) following injury-reperfusion (I/R) AKI, and at day 3 (D3) following I/R AKI.

FIG. 7B is a table showing greater mortality in AKI induced rats administered SDF-1 knockdown MSCs when compared to control MSCs.

### DETAILED DESCRIPTION OF THE INVENTION

The present invention provides a method for assaying mesenchymal stromal cells for their therapeutic effectiveness. The invention is based upon the finding that the knock down of the stromal derived factor-1 (SDF-1) and vascular endothelial growth factor (VEGF) each independently decreases the protective effect of MSCs against kidney injury.

In bone marrow transplantation, the chemokine SDF-1 (CXCL12) mediates recruitment to and engraftment in the bone marrow niches of CXCR4-expressing hematopoietic stem cells (HSC). SDF-1 is secreted by resident MSCs acting as a survival factor in both hematopoietic stem cells (HSCs) and MSCs. It has been shown that ischemia/reperfusion induced (IRI) acute kidney injury (AKI) in rats and mice causes robust up regulation of SDF-1 and CXCR4 in tubular cells, and that renal SDF-1 is a principal homing signal for CXCR4-expressing MSC and other cells. Since it has been demonstrated that allogeneic MSCs protect renal function and stimulate organ repair in rats and in study subjects, we investigated whether knock down (siRNA) of SDF-1 expression and secretion by MSC alters their kidney protective actions when compared to wild type MSC in rats with IRI AKI. We found that SDF-1 expression and release by infused MSC in AKI is important to their renoprotective activity.

Vascular damage is an early and important mediator of AKI, and also leads to long-term damage and progressive loss of renal function. VEGF is the major angiogenic factor that is important for vascular maintenance after AKI. Renal ischemia inhibits VEGF expression by multiple mechanisms, shifting the balance from a pro-angiogenic to an anti-angiogenic milieu, thereby inhibiting renal repair and paving the way to long-term progressive loss of renal function. MSCs express VEGF amongst other growth factors and have been shown to exert paracrine actions that are renoprotective and enhance recovery from AKI. Recently, IGF-1 has been implicated as a paracrine mediator of renoprotection in a cisplatinum model of AKI. Because a single factor is unlikely to be the sole mediator of renoprotection, we examined the potential significance of VEGF as a renoprotective mediator of MSCs in AKI. Accordingly, VEGF was knocked down in MSCs and their organ protection activity in AKI was compared to that of wild type MSCs. Our data show that knocking down VEGF secretion in MSCs decreases the proliferative effect in rat proximal tubular cells in vitro and decreases their effectiveness after AKI in vivo. Rats treated with VEGF knockdown MSCs had a higher mortality and slower recovery of renal function after AKI. These data clearly demonstrate the importance of VEGF mediating renoprotection of MSCs after AM. Furthermore, microvessel density was significantly higher in animals treated with regular MSCs compared to VEGF knockdown MSCs and controls, demonstrating the importance of early VEGF administration via MSCs for the long-term outcome after AKI.

Basile has demonstrated that VEGF is down regulated after AKI and a long-term consequence of AKI is decreased microvessel density and impaired renal concentrating ability (Basile D P, et al. Am J Physiol Renal Physiol. 2001; 281:F887-99; Basile D P, et al. Am J Physiol Renal Physiol. 2008; 294:F928-36). MSC treatment early in the course of AKI might appear thus beneficial for the long-term outcome after AKI.

Based on these findings, an assay was developed to detect the levels of SDF-1 and VEGF in MSCs to predict the therapeutic effectiveness of any given cell population of culture. These assays allow for the repeated safe use of cultured MSCs that have been passaged, expanded, and/or frozen and thawed. Thus, use of the assay expands the safe use of MSCs to expanded and frozen cell cultures.

MSCs may be passaged or expanded according to any methods known in the art. Specific passaging protocols are provided in the examples below. Likewise, MSCs may be frozen and/or thawed according to any method known in the art. Specific freezing/thawing protocols are provided in the examples below.

Moreover, the expression of SDF-1 and/or VEGF may be measured by any method known in the art. These methods include measuring amounts of mRNA or protein. Protein measurement methods include Western blotting, FACS and ELISA. mRNA measurement methods include northern blotting and rtPCR.

In specific embodiments, the amounts of SDF-1 and/or VEGF that are secreted into the media by cultured MSCs are measured in order to determine the expression of SDF-1 and/or VEGF. Optionally, MSCs are cultured in media with serum until they reach a sufficient density for harvesting for measurement of protein expression. The media with serum is then removed from the MSCs and replaced with serum free medium. The cells are allowed to secrete SDF-1 and/or VEGF into the serum free medium for a period of time. In certain preferred embodiments, this period of time is 6, 12, 18, 24 or 48 hours or longer. The amount of SDF-1 and/or VEGF is then assayed in the serum free media in order to measure the expression of SDF-1 and/or VEGF. The amount of SDF-1 and/or VEGF in the medium can be measured using ELISA, Western blot or other techniques known in the art. In other embodiments, the ELISA test is performed in a well in a polystyrene microtiter plate, cassette, or on a dipstick.

In other embodiments, a variant of ELISA, the enzyme-linked coagulation assay or ELCA (U.S. Pat. No. 4,668,621 incorporated herein by reference in its entirety) is used. In this system, the reactions can be performed at physiological pH in the presence of a wide variety of buffers.

In specific embodiments, the expression of SDF-1 and/or VEGF is compared between a population of MSCs that have been passaged and/or frozen and thawed and a fresh population of MSCs. A fresh population of MSCs is a population that has been isolated from a subject, but has not been passaged, expanded or frozen. Comparisons can also be made between MSC populations that have been passaged different numbers of times. For example, MSCs of passage 1, 2, 3, 4, 5, 6, 7, 8, 9 or 10 may be compared to MSCs that are fresh or of passage 1, 2, 3, 4, 5, 6, 7, 8 or 9. Likewise, MSCs of any passage or fresh MSCs that have never been frozen could be compared to MSCs of any passage or fresh MSCs that have been frozen and thawed.

When passaged and/or frozen and thawed MSC populations are compared to fresh MSC populations, the passaged and/or frozen and thawed cells are therapeutically effective if the expression of SDF-1 and/or VEGF is similar to the expression of SDF-1 and/or VEGF in the fresh MSCs. In some embodiments, when the expression of SDF-1 in a passaged and/or frozen and thawed MSC population is 75%, 80%, 85%, 90%, 95%, or greater than 100% of expression of SDF-1 in a fresh MSC population, this means that the passaged and/or frozen and thawed MSC population is therapeutically effective. Likewise, in some embodiments, when the expression of VEGF in a passaged and/or frozen and thawed MSC population is 75%, 80%, 85%, 90%, 95%, or greater than 100% of expression of VEGF in a fresh MSC population, this means that the passaged and/or frozen and thawed MSC population is therapeutically effective. Also, in some embodiments, when the expression of VEGF and SDF-1 in a passaged and/or frozen and thawed MSC population are each independently 75%, 80%, 85%, 90%, 95%, or greater than 100% of expression of VEGF and SDF-1 in a fresh MSC population, this means that the passaged and/or frozen and thawed MSC population is therapeutically effective.

In other embodiments, when passaged and/or frozen and thawed MSCs have lower SDF-1 and/or VEGF expression than fresh MSCs, the dose of passaged and/or frozen and thawed cells could be increased to make up for the deficiency. For example, if passaged MSCs had SDF-1 and/or VEGF expression that was 50% of fresh MSCs, then twice the dose of passaged cells would be used compared to the effective dose of fresh MSCs.

In other embodiments, the assays of the invention are used to maintain a constant dose of MSCs that are SDF-1 and/or VEGF positive in passaged and/or frozen and thawed MSCs when compared to fresh MSCs. For example, if a fresh population of MSCs was 90% SDF-1 positive and a passaged population of MSCs was 45% positive, twice as many passaged MSCs as fresh MSCs could be administered to provide the same number of SDF-1 positive MSCs.

According to certain embodiments of the invention, other MSC markers are also measured. For example, the presence of CD105 and/or CD90 is measured in some embodiments. In other embodiments, the absence of CD34 and/or CD45 is measured. The presence of CD105 and/or CD90 as well as the absence of CD34 and/or CD45 is indicative of the MSC phenotype. In other embodiments, adipogenic, osteogenic and/or chondrigenic assays are used to show that the MSCs possess the characteristic ability of trilinieage differentiation.

Methods of Producing Mesenchymal Stromal Cells

In certain embodiments, the mesenchymal stromal cells (MSCs) of the invention are cultured in media supplemented with platelet lysate (PL) or fetal calf serum (FCS). In one embodiment of the method of producing MSCs of the invention, the starting material for the MSCs is bone marrow isolated from healthy donors. Preferably, these donors are mammals. More preferably, these mammals are humans. In one embodiment of the method of producing MSCs of the invention, the bone marrow is cultured in tissue culture flasks between 2 and 10 days prior to washing non-adherent cells from the flask. Optionally, the number of days of culture of bone marrow cells prior to washing non-adherent cells is 2 to 3 days. Preferably the bone marrow is cultured in platelet lysate (PL) containing media. For example, 300 μl of bone marrow is cultured in 15 ml of PL supplemented medium in T75 or other adequate tissue culture dishes.

After washing away the non-adherent cells, the adherent cells are also cultured in media that has been supplemented with platelet lysate (PL) or FCS. Thrombocytes are a well characterized human product which already is widely used in clinics for patients in need. Thrombocytes are known to produce a wide variety of factors, e.g. PDGF-BB, TGF-β, IGF-1, and VEGF. In one embodiment of the method of producing MSCs of the invention, an optimized preparation of PL is used. This optimized preparation of PL is made up of pooled platelet rich plasmas (PRPs) from at least 10 donors (to equalize for differences in cytokine concentrations) with a minimal concentration of 3×10⁹ thrombocytes/ml.

According to preferred embodiments of the method of producing MSCs of the invention, PL was prepared either from pooled thrombocyte concentrates designed for human use (produced as TK5F from the blood bank at the University Clinic UKE Hamburg-Eppendorf, Germany pooled from 5 donors) or from 7-13 pooled buffy coats after centrifugation with 200×g for 20 min. Preferably, the PRP was aliquoted into small portions, frozen at −80° C., and thawed immediately before use. PL-containing medium was prepared freshly for each cell feeding. In a preferred embodiment, medium contained αMEM as basic medium supplemented with 5 IU Heparin/ml medium (source: Ratiopharm) and 5% of freshly thawed PL. The method of producing MSCs of the invention, uses a method to prepare PL that differs from others according to the thrombocyte concentration and centrifugation forces. The composition of this PL is described in greater detail, below.

In one embodiment of the method of producing MSCs, the adherent cells are cultured in PL-supplemented media at 37° C. with approximately 5% CO₂ under hypoxic conditions. Preferably, the hypoxic conditions are an atmosphere of 5% O₂. In some situations hypoxic culture conditions allow MSCs to grow more quickly. This allows for a reduction of days needed to grow the cells to 90-95% confluence. Generally, it reduces the growing time by three days. In another embodiment of the method of producing MSCs of the invention, the adherent cells are cultured in PL-supplemented media at 37° C. with approximately 5% CO₂ under normoxic conditions, i.e. wherein the O₂ concentration is the same as atmospheric O₂, approximately 20.9%. Preferably, the adherent cells are cultured between 9 and 12 days, being fed every 3-4 days with PL-supplemented media. In one embodiment of the method of producing MSCs of the invention, the adherent cells are grown to between 90 and 95% confluence. Preferably, once this level of confluence is reached, the cells are trypsinized to release them from the plate for subsequent passage.

In certain embodiments, the population of cells that is isolated from the plate is between 50-99% MSCs. In other embodiments, isolated MSCs are enriched in MSCs so that 25%, 30%, 35%, 40%, 45%, 50%, 55%, 60%, 65%, 70%, 75%, 80%, 85%, 90%, 95%, 96%, 97%, 98% or 99% of the cell population are MSCs. In other embodiments, the MSCs are greater than 95% of the isolated cell population.

In another embodiment of the method of producing MSCs of the invention, the cells are frozen after they are released from the tissue culture plate. Freezing is performed in a step-wise manner in a physiologically acceptable carrier, 5 to 10% human serum albumin and 10% DMSO. Thawing is also performed in a step-wise manner. Preferably, when thawed, the frozen MSCs of the invention are diluted 4:1 to remove DMSOIn this case, frozen MSCs of the invention are thawed quickly at 37° C. and administered intravenously without any dilution or washings. Optionally the cells are administered following any protocol that is adequate for the transplantation of hematopoietic stromal cells (HSCs). Preferably, the serum albumin is human serum albumin.

In another embodiment of the method of producing MSCs of the invention, the cells are frozen in aliquots of 10⁴-10¹² cells in 50 mL of physiologically acceptable carrier and human serum albumin (HSA). In another embodiment of the method of producing MSCs of the invention, the cells are frozen in aliquots of 10⁶-10⁸ cells in 50 mL of physiologically acceptable carrier and human serum albumin (HSA). In another embodiment of the method of producing MSCs of the invention, the cells are frozen in aliquots of 10⁶-10⁸ cells per kg of subject body weight, in 50 mL of physiologically acceptable carrier and serum albumin (HSA). In one aspect of these embodiments, when a therapeutic dose is being assembled, the appropriate number of cryovials is thawed in order to thaw the appropriate number of cells for the therapeutic dose. Preferably, after DMSO is diluted from the thawed cells, the number of cryovials chosen is placed in a sterile infusion bag with 5-10% human serum albumin. Once in the bag, the MSCs do not aggregate and viability remains greater than 95% even when the MSCs are stored at room temperature for at least 6 hours. This provides ample time to administer the MSCs of the invention to a patient in an operating room. Optionally, the physiologically acceptable carrier is Plasma-lyte. Preferably the serum albumin is human serum albumin. Preferably the albumin is present at a concentration of 5% w/v. Suspending the 10⁶-10⁸ cells MSCs of the invention in greater than 40 mL of physiological carrier is critical to their biological activity. If the cells are suspended in lower volumes, the cells are prone to aggregation. Administration of aggregated MSCs to mammalian subjects has resulted in cardiac infarction. Thus, it is crucial that non-aggregated MSCs be administered according to the methods of the invention. The presence of albumin is also critical because it prevents aggregation of the MSCs and also prevents the cells from sticking to plastic containers the cells pass through when administered to subjects.

In another embodiment of the method of producing MSCs of the invention, a closed system is used for generating and expanding the MSCs of the invention from bone marrow of normal donors. This closed system is a device to expand cells ex vivo in a functionally closed system. In one specific embodiment, the closed system includes: 1. a central expansion unit preferably constructed similarly to bioreactors with compressed (within a small unit), but extended growth surfaces; 2. media bags which can be sterilely connected to the expansion unit (e.g. by welding tubes between the unit and the bags) for cell feeding; and 3. electronic devices to operate and monitor automatically the medium exchange, gas supply and temperature.

The advantages of the closed system in comparison to conventional flask tissue culture are the construction of a functionally closed system, i.e. the cell input and media bags are sterile welded to the system. This minimizes the risk of contamination with external pathogens and therefore may be highly suitable for clinical applications. Furthermore, this system can be constructed in a compressed form with consistently smaller cell culture volumes but preserved growth area. Also the closed system saves costs for the media and the whole expansion process.

The construction of the closed system may involve two sides: the cells are grown inside of multiple fibres with a small medium volume. In some embodiments, the culture media contains growth factors for growth stimulation, and medium without expensive supplements is passed outside the fibres. The fibres are designed to contain nanopores for a constant removal of potentially growth-inhibiting metabolites while important growth-promoting factors are retained in the growth compartment.

In certain embodiments of the method of producing MSCs of the invention, the closed system is used in conjunction with a medium for expansion of MSCs which does not contain any animal proteins, e.g. fetal calf serum (FCS). FCS has been connected with adverse effects after in vivo application of FCS-expanded cells, e.g. formation of anti-FCS antibodies, anaphylactic or Arthus-like immune reactions or arrhythmias after cellular cardioplasty. FCS may introduce unwanted animal xenogeneic antigens, viral, prion and zoonose contaminations into cell preparations making new alternatives necessary.

Methods of Using Mesenchymal Stromal Cells

The MSCs subject to the assay of the invention are used to treat or ameliorate conditions including, but not limited to, stroke, multi-organ failure (MOF), acute renal failure (ARF) of native kidneys, ARF of native kidneys in multi-organ failure, ARF in transplanted kidneys, kidney dysfunction, multi-organ dysfunction and wound repair refer to conditions known to one of skill in the art. Descriptions of these conditions may be found in medical texts, such as The Kidney, by Barry M. Brenner and Floyd C. Rector, Jr., WB Saunders Co., Philadelphia, last edition, 2001, which is incorporated herein in its entirety by reference.

Stroke or cerebral vascular accident (CVA) is a clinical term for a rapidly developing loss of brain function, due to lack of blood supply. The reason for this disturbed perfusion of the brain can be thrombosis, embolism or hemorrhage. Stroke is a medical emergency and the third leading course of death in Western countries. It is predicted that stroke will be the leading cause of death by the middle of this century. These factors for stroke include advanced age, previous stroke or ischemic attack, high blood pressure, diabetes mellitus, high cholesterol, cigarette smoking and cardiac arrhythmia with atrial fibrillation. Therefore, a great need exists to provide a treatment for stroke patients.

ARF is defined as an acute deterioration in renal excretory function within hours or days. In severe ARF, the urine output is absent or very low. As a consequence of this abrupt loss in function, azotemia develops, defined as a rise of serum creatinine and blood urea nitrogen levels. Serum creatinine and blood urea nitrogen levels are measured. When these levels have increased to approximately 10 fold their normal concentration, this corresponds with the development of uremic manifestations due to the parallel accumulation of uremic toxins in the blood. The accumulation of uremic toxins causes bleeding from the intestines, neurological manifestations most seriously affecting the brain, leading, unless treated, to coma, seizures and death. A normal serum creatinine level is about 1.0 mg/dL, a normal blood urea nitrogen level is about 20 mg/dL. In addition, acid (hydrogen ions) and potassium levels rise rapidly and dangerously, resulting in cardiac arrhythmias and possible cardiac standstill and death. If fluid intake continues in the absence of urine output, the patient becomes fluid overloaded, resulting in a congested circulation, pulmonary edema and low blood oxygenation, thereby also threatening the patient's life. One of skill in the art interprets these physical and laboratory abnormalities, and bases the needed therapy on these findings.

Multi-organ Failure (MOF) is a condition in which kidneys, lungs, liver and heart functions are generally impaired simultaneously or successively, resulting in mortality rates as high as 100% despite the conventional therapies utilized to treat ARF. These patients frequently require intubation and respirator support because their lungs develop Adult Respiratory Distress Syndrome (ARDS), resulting in inadequate oxygen uptake and CO₂ elimination. MOF patients also depend on hemodynamic support, vasopressor drugs, and occasionally, an intra-aortic balloon pump, to maintain adequate blood pressures since these patients are usually in shock and suffer from heart failure. There is no specific therapy for liver failure which results in bleeding and accumulation of toxins that impair mental functions. Patients may need blood transfusions and clotting factors to prevent or stop bleeding. MOF patients will be given stem cell therapy when the physician determines that therapy is needed based on assessment of the patient.

Early graft dysfunction (EGD) or transplant associated-acute renal failure (TA-ARF) is ARF that affects the transplanted kidney in the first few days after implantation. The more severe TA-ARF, the more likely it is that patients will suffer from the same complications as those who have ARF in their native kidneys, as above. The severity of TA-ARF is also a determinant of enhanced graft loss due to rejection(s) in the subsequent years. These are two strong indications for the prompt treatment of TA-ARF with the stem cells of the present invention.

Chronic renal failure (CRF) or Chronic Kidney Disease (CKD) is the progressive loss of nephrons and consequent loss of renal function, resulting in End Stage Renal Disease (ESRD), at which time patient survival depends on dialysis support or kidney transplantation. Need for stem cell therapy of the present invention will be determined on the basis of physical and laboratory abnormalities described above.

In some embodiments of methods of use of MSCs subject to the assay of the invention, the MSCs subject to the assay of the invention are administered to patients in need thereof when one of skill in the art determines that conventional therapy fails. Conventional therapy includes hemodialysis, antibiotics, blood pressure medication, blood transfusions, intravenous nutrition and in some cases, ventilation on a respirator in the ICU. Hemodialysis is used to remove uremic toxins, improve azotemia, correct high acid and potassium levels, and eliminate excess fluid. In other embodiments of methods of use of MSCs of the invention, the MSCs of the invention are administered as a first line therapy. The methods of use of MSCs of the present invention is not limited to treatment once conventional therapy fails and may also be given immediately upon developing an injury or together with conventional therapy.

In certain embodiments, the MSCs subject to the assay of the invention are administered to a subject once. This one dose is sufficient treatment in some embodiments. In other embodiments the MSCs subject to the assay of the invention are administered 2, 3, 4, 5, 6, 7, 8, 9 or 10 times in order to attain or sustain a therapeutic effect.

Monitoring patients for a therapeutic effect of the stem cells delivered to a patient in need thereof and assessing further treatment will be accomplished by techniques known to one of skill in the art. For example, renal function will be monitored by determination of blood creatinine and BUN levels, serum electrolytes, measurement of renal blood flow (ultrasonic method), creatinine and inulin clearances and urine output. A positive response to therapy for ARF includes return of excretory kidney function, normalization of urine output, blood chemistries and electrolytes, repair of the organ and survival. For MOF, positive responses also include improvement in blood pressure and improvement in functions of one or all organs.

In other embodiments the MSCs subject to the assay of the invention are used to effectively repopulate dead or dysfunctional kidney cells in subjects that are suffering from chronic renal pathology including chronic renal failure because of the “plasticity” of the MSC populations. The term “plasticity” refers to the phenotypically broad differentiation potential of cells that originate from a defined stem cell population. MSC plasticity can include differentiation of stem cells derived from one organ into cell types of another organ. “Transdifferentiation” refers to the ability of a fully differentiated cell, derived from one germinal cell layer, to differentiate into a cell type that is derived from another germinal cell layer.

It was assumed, until recently, that stem cells gradually lose their pluripotency and thus their differentiation potential during organogensis. It was thought that the differentiation potential of somatic cells was restricted to cell types of the organ from which respective stem cells originate. This differentiation process was thought to be unidirectional and irreversible. However, recent studies have shown that somatic stem cells maintain some of their differentiation potential. For example, stromal cells may be able to transdifferentiate into muscle, neurons, liver, myocardial cells, and kidney. It is possible that as yet undefined signals that originate from injured and not from intact tissue act as transdifferentiation signals.

In certain embodiments, a therapeutically effective dose of MSCs is delivered to the patient. An effective dose for treatment will be determined by the body weight of the patient receiving treatment, and may be further modified, for example, based on the severity or phase of the stroke, kidney or other organ dysfunction, for example the severity of ARF, the phase of ARF in which therapy is initiated, and the simultaneous presence or absence of MOF. In some embodiments of the methods of use of the MSCs of the invention, from about 1×10⁵ to about 1×10¹⁰ MSCs per kilogram of recipient body weight are administered in a therapeutic dose. Preferably from about 1×10⁵ to about 1×10⁸ MSCs per kilogram of recipient body weight is administered in a therapeutic dose. More preferably from about 7×10⁵ to about 5×10¹⁰ MSCs per kilogram of recipient body weight is administered in a therapeutic dose. More preferably from about 1×10⁶ to about 1×10⁸ MSCs per kilogram of recipient body weight is administered in a therapeutic dose. More preferably from about 7×10⁵ to about 7×10⁶ MSCs per kilogram of recipient body weight is administered in a therapeutic dose. More preferably about 2×10⁶ MSCs per kilogram of recipient body weight is administered in a therapeutic dose. The number of cells used will depend on the weight and condition of the recipient, the number of or frequency of administrations, and other variables known to those of skill in the art. For example, a therapeutic dose may be one or more administrations of the therapy.

The therapeutic dose of stem cells are administered in a suitable solution for injection. Solutions are those that are biologically and physiologically compatible with the cells and with the recipient, such as buffered saline solution, Plasma-lyte or other suitable excipients, known to one of skill in the art.

In certain embodiments of the MSCs of the invention are administered to a subject at a rate between approximately 0.5 and 1.5 mL of MSCs in physiologically compatible solution per second. Preferably, the MSCs of the invention are administered to a subject at a rate between approximately 0.83 and 1.0 mL per second. More preferably, the MSCs are suspended in approximately 50 mL of physiologically compatible solution and is completely injected into a subject between approximately one and three minutes. More preferably the 50 mL of MSCs in physiologically compatible solution is completely injected in approximately one minute.

In other embodiments, the MSCs are used in trauma or surgical patients scheduled to undergo high risk surgery such as the repair of an aortic aneurysm. Administration of MSCs of the invention to these patients for prophylactic MSC collection and preparation prior to major surgery. In the case of poor outcome, including infected and non-healing wounds, development of MOF post surgery, the patient's own MSCs, prepared according to the methods of the invention, that are cryopreserved may be thawed out and administered as detailed above. Patients with severe ARF affecting a transplanted kidney may either be treated with MSCs, prepared according to the methods of the invention, from the donor of the transplanted kidney (allogeneic) or with cells from the recipient (autologous). Allogeneic or autologous MSCs, prepared according to the methods of the invention, are an immediate treatment option in patients with TA-ARF and for the same reasons as described in patients with ARF of their native kidneys.

In certain embodiments, the MSCs of the invention are administered to the patient by infusion intravenously or intra-arterially (via femoral artery into supra-renal aorta). Preferably, the MSCs of the invention are administered via the supra-renal aorta. In certain embodiments, the MSCs of the invention are administered through a catheter that is inserted into the femoral artery at the groin. Preferably, the catheter has the same diameter as a 12-18 gauge needle. More preferably, the catheter has the same diameter as a 15 gauge needle. The diameter is relatively small to minimize damage to the skin and blood vessels of the subject during MSC administration. Preferably, the MSCs of the invention are administered at a pressure that is approximately 50% greater than the pressure in the subject's aorta. More preferably, the MSCs of the invention are administered at a pressure of between about 120 and 160 psi. The shear stressed created by the pressure of administration does not cause injury to the MSCs of the invention. Generally, at least 95% of the MSCs of the invention survive injection into the subject. Moreover, the MSCs are generally suspended in a physiologically acceptable carrier containing about 5% HSA. The HSA, along with the concentration of the cells prevents the MSCs from sticking to the catheter or the syringe, which also insures a high (i.e. greater than 95%) rate of survival of the MSCs when they are administered to a subject. The catheter is advanced into the supra-renal aorta to a point approximately 20 cm above the renal arteries. Preferably, blood is aspirated to verify the intravascular placement and to flush the catheter. More preferably, the position of the catheter is confirmed through a radiographic or ultrasound based method. Preferably the methods are transesophageal echocardiography (TEE) or an X-ray. The MSCs of the invention are then transferred to a syringe which is connected to the femoral catheter. The MSCs, suspended in the physiologically compatible solution are then injected over approximately one to three minutes into the patient. Preferably, after injection of the MSCs of the invention, the femoral catheter is flushed with normal saline. Optionally, the pulse of the subject found in the feet is monitored, before, during and after administration of the MSCs of the invention. The pulse is monitored to ensure that the MSCs do not clump during administration. Clumping of the MSCs can lead to a decrease or loss of small pulses in the feet of the subject being administered MSCs.

### EXAMPLES

### Example 1

### MSC Potency Assay Protocol

This protocol involves serial passaging and assaying for SDF-1 and VEGF at passages 0 through 6 (P0-P6) of mesenchymal stromal cells (MSCs). SDF-1 and VEGF are assayed at both the transcription and translation levels by use of rtPCR and ELISA assays. Characteristic MSC markers and differentiation abilities are determined by standard procedures. At passage 1 (P1), passage 3 (P3) and passage 6 (P6), MSCs are cryopreserved, then thawed and assayed as described in greater detail below.

MSCs are grown to 70-90% confluence in medium containing platelet rich plasma (PRP) at each passage as shown on the Overview of Phase II Dose Production. At 70-90% confluence, the MSCs are divided into 2 groups. The first group is trypsinized and used for passaging and the second group (a defined cell number) is assayed.

The medium from the second group is sampled for SDF-1 and VEGF content using ELISA. Cells from the second group are also removed and assayed as follows:
(a) rtPCR is performed using SDF-1 and VEGF primers.
(b) FACS analysis is performed to demonstrate +CD105, +CD90, −CD34, −CD45, HLA-DR
(c) Adipo-, osteo- and chondrogenic differentiation assays are performed with demonstration of differentiation potential noted.

### Example 2

### Creating MSC for a Master Cell Bank

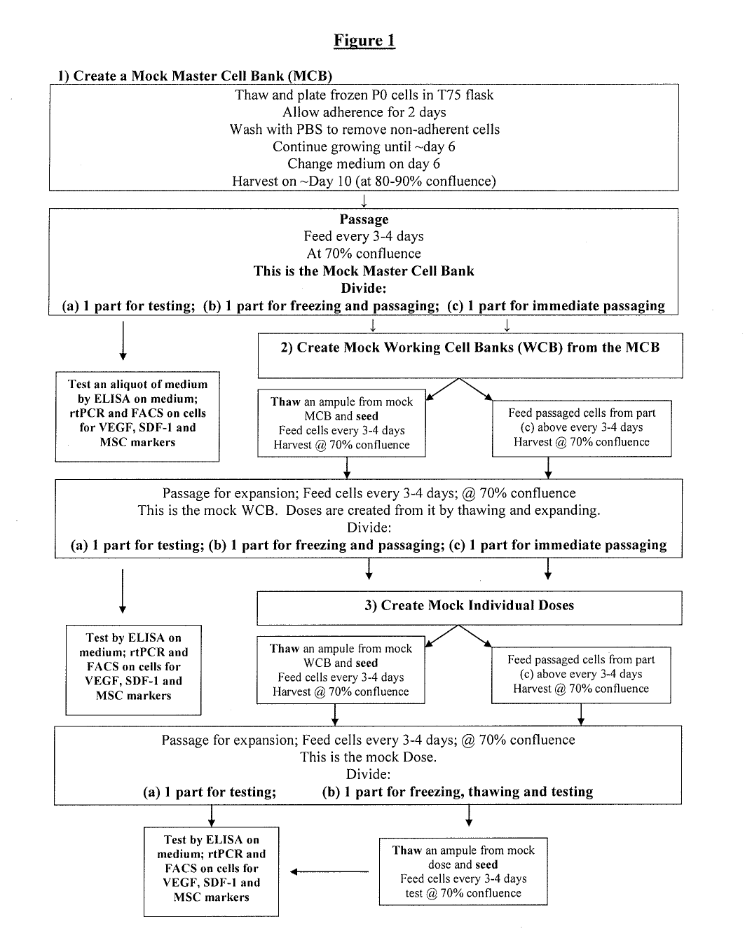

This MSC protocol is summarized in FIG. 1.

Creating a Master Cell Bank (MCB)

Unpassaged (P0) MSCs are thawed and plated in two T75 flasks containing media supplemented with 5% platelet lysate. The MSCs are allowed to adhere to the flasks for 2 days, and then the flasks are washed with phosphate buffered saline (PBS) to remove non-adherent cells. The cells continue to be grown until day 6 when the media are changed. The MSCs are harvested when they reach 70-90% confluence.

The cells are then passaged and fed every 3-4 days. The cells are harvested when they reach 70%-90% confluence. The harvest of this first passage is referred to as the master cell bank.

The master cell bank is split into three portions. The first portion is for testing, the second is for freezing and passaging and the third is for immediate passaging. The first portion for testing is tested by ELISA, rtPCR and FACS for VEGF, SDF-1 and MSC markers as described in Example 1.

Creating Working Cell Banks (WCBs) from the Master Cell Bank (MCB)

When creating a working cell bank from a frozen master cell bank portion, the cells are thawed and fed every 3-4 days until they reach 70-90% confluence. If the cells are from a master cell bank portion prepared for immediate passaging, the cells need only be fed every 3-4 days until 70-90% confluent. These cells are then harvested at 70-90% confluence making up the working cell bank. Doses are created from the working cell bank by thawing and expanding the MSCs.

The working cell bank is split into three portions. The first portion is for testing, the second is for freezing and passaging and the third is for immediate passaging. The first portion for testing is tested by ELISA, rtPCR and FACS for VEGF, SDF-1 and MSC markers as described in Example 1.

Creating Individual Doses from the Working Cell Bank (WCB) or Master Cell Bank (MCB)

When creating a working cell bank/product dose from a frozen master cell bank portion, the cells are thawed and fed every 3-4 days until they reach 70-90% confluence. If the cells are from a master cell bank portion prepared for immediate passaging, the cells need only be fed every 3-4 days until 70-90% confluent. These cells are then harvested at 70-90% confluence making up the working cell bank. Doses are created from the working cell bank by thawing and expanding the MSCs or directly from the master cell bank.

The individual doses are split into two portions. The first portion is for testing, the second is for freezing, thawing and administration or testing. Any portion used for testing is grown in serum free media for 24 hours and tested by ELISA, rtPCR and FACS for VEGF, SDF-1 and MSC markers as described in Example 1.

### Example 3

### SDF-1 Knock-Down in Mesenchymal Stromal Cells (MSCs)

SDF-1 Knock Down in Cultured Rat MSC

All experiments were done using wt F344 rat mesenchymal stromal cells (MSCs) at passage 3 or 4. MSCs were cultured in DMEM-F12 (Sigma) +10% FBS (HyClone) medium using standard procedures. SDF-1 knock down was achieved using the SiPORT™ NeoFX™ kit (Ambion). A 12 well plate system and 30nM SDF-1 siRNA/well with 12 μL NeoFX transfection agent/well was used.

Cultured MSCs were trypsinized, harvested and resuspended in normal growth medium at a concentration of 1×10⁵ cells/ml. Cells were incubated with a mix of 2 different siRNA for SDF-1 at a final concentration of 30 nM siRNA/well plus 12 μL transfection agent/well. Cells were then plated at 1×10⁵ cells/well in 12 well culture plates and cultured 24 hrs at 37° C. The growth medium was replaced after 24 hrs with standard growth medium.

SDF-1 knock down was confirmed at both protein and RNA level by ELISA and PCR techniques.

Protein Assay

On days 2, 3, and 4 the growth medium was collected and analyzed by ELISA (Quantikine CXCL12/SDF-1α kit; R&D Systems) for protein SDF-1 concentration. By day 4, protein levels of SDF-1 were found to be reduced by approximately 50% in the medium.

PCR Assay

MSCs that had been cultured and transfected with SDF-1 siRNA were harvested at 2, 3 and 4 days post transfection. rtPCR assays to determine SDF-1 mRNA levels were performed on these cells and compared with non-transfected cells. After 72 hrs, SDF-1 RNA levels were reduced approximately 20 fold in transfected cells.

At 4 days post knock down, with reference to 2 housekeeping genes gene regulation for SDF-1 was down by a factor of 5.2, absolute gene regulation was decreased by 2.4.

Induction of AKI and MSC Treatment

All SDF-1 knock-down MSCs that were used in in vivo experiments had been transfected in a 12 well system using 12 μL transfection agent/well, and 30 nM SDF-1 siRNA/well, washed after 24 hrs and cultured 4 days post transfection, as those were the conditions that maximized SDF-1 knockdown.

In vivo experiments were carried out in wt F344 female rats weighing between 150 and 200 g. 3 groups of 5 to 7 animals were anesthetized and subjected to 42 min bilateral renal pedicle clamping (I/R AKI). Immediately upon reperfusion, rats were treated with one of the following: vehicle (Serum free medium, 1 ml delivered via the left carotid artery; n=7); normal MSCs (control; 2×10⁶ cells/kg body weight suspended in 1 ml serum free medium and delivered via the left carotid artery; n=6); or SDF-1 knockdown MSCs (knockdown achieved as described above; 2×10⁶ cells/kg body weight suspended in 1 ml serum free medium and delivered via the left carotid artery; n=5).

Animals were allowed to recover, and renal function as assessed by SCr levels was checked on post-op days 1 and 3 and compared with baseline values. Serum creatinine (SCr) levels one day post I/R AKI in MSC treated rats were approximately ⅓ lower than those of vehicle treated rats. By contrast, SCr levels in SDF-1 knockdown MSC treated rats were comparable to those of vehicle treated rats one day post I/R AKI. Plasma SDF-1 levels in all three groups were similar and remained stable. Urine SDF-1 levels (normalized to creatinine) are significantly increased at 3 and 5 hrs, and 1 day post I/R AKI in SDF-1 kd MSC treated rats as compared to normal MSC treated rats.

Mortality: 1/7 of the vehicle treated rats died by post I/R AKI day 3. 0/6 (none) of the control MSC treated rats died. 2/5 of the SDF-1 knock-down MSC treated rats died by post I/R AKI day 3, further indicating that reducing MSC derived SDF-1 levels inhibits the ability of MSC to protect kidneys from ischemic renal injury and animals from dying.

### Example 4

### VEGF Knock-Down in Mesenchymal Stromal Cells (MSCs)

Animals and Cells

The Institutional Animal Use and Care Committees (IACUC) of the Veterans Affairs Medical Center (Salt Lake City, Utah, USA) approved all procedures involving animals. MSCs were generated from F344 rats as described before Togel F, et al., Am J Physiol Renal Physiol. 2005; 289: F31-42. In brief, femurs of sacked animals were flushed with PBS and cells cultured in alpha-MEM containing 10% FBS. Adherent cells were removed after 3 days and MSCs passaged at subconfluence. FACS staining for CD45, CD90 and CD105 and differentiation into adipocytes, osteocytes and chondrocytes characterized MSCs.

Surgical Procedures and MSC Treatment

Ischemia/reperfusion acute kidney injury (AKI) was induced in anesthetized female Sprague Dawley (SD) rats. In brief, renal pedicles of adult female SD rats weighting 200-250 g were clamped for 48 min. and animals were infused immediately after reflow and via the left carotid artery with 2×10⁶/kg body weight MSCs derived from F344 rats (wild type or VEGF siRNA treated) in 1 ml of PBS. All controls with identical AKI were infused, via the left carotid artery, with 1 ml of PBS. This constitutes an allogeneic MSC protocol.

Kidney Function

Serum creatinine was determined using the Dimension RxL Max Clinical Chemistry System (Dade Behring, Deerfield, Ill., USA) from a plasma sample of heparinized blood.

VEGF siRNA Knockdown

Cultured MSCs from F344 rats were treated with siRNA targeted at three different exons of the VEGF gene that are common to all splice variants (exons 2-6) and NeoFx transfection agent (Ambion, Austin, Tex., USA). Silencer® pre-designed siRNAs (siRNA ID #192613, 192614 and 192615) were purchased (Ambion) and tested at three different concentrations (5, 10, 30 nm) in regular culture medium. Cells were incubated for 24 hrs with siRNA and washed with PBS afterwards. A concentration of 10 nm proved to be most effective and was therefore used for all subsequent experiments. Controls consisted of cells treated with Silencer® negative control siRNA (Ambion), NeoFx transfection agent only and untreated cells. Gene expression measured by real-time quantitative RT-PCR with a SmartCycler (Cepheid, Sunnyvale, Calif., USA) was tested 24 and 48 hrs after knockdown (VEGF forward primer: gcactggaccctggcttt (SEQ ID NO:1); reverse primer: cggggtactcctggaagatg) (SEQ ID NO:2), and VEGF protein secretion by ELISA in medium conditioned for 24 hrs (RnD Systems, Minneapolis, Minn., USA). VEGF-receptor primers used were: flt-1: forward—agcaacaggtgcaggaaacca (SEQ ID NO:3); reverse—tgcaccgaatagcgagcaga (SEQ ID NO:4); flt-4 forward—ctccaacttcttgcgtgtca (SEQ ID NO:5); reverse—acaaggtcctccatggtcag; (SEQ ID NO:6) flk-1: caggggagggttggcataga (SEQ ID NO:5); reverse—caccccagatcggtgagaaag (SEQ ID NO:5).

In Vitro Studies

Rat proximal tubular cells (NRK, ATCC, Manassas, Va.) were seeded in 96-well plates at a density of 15,000 cells/well and subjected to 48 hrs of stimulation either with conditioned medium from MSCs or serum-free control medium or medium containing 10% FBS (Hyclone, Logan, Utah, USA). Conditioned medium was generated from 1×10⁶ MSCs seeded in a well of a 6-well plate over 24 hrs. Proliferative activity was determined using a colorimetric tetrazolium based MTT assay.

Microvessel Density Determination

Kidney sections of SD rats 4 weeks after induction of AKI were immunostained with mouse monoclonal CD34 antibody (Santa Cruz, Santa Cruz, Calif., USA) to visualize microvessels. No nuclear counterstaining was applied. The percentage area of stained microvessel was determined with ImageJ (National Institutes of Health) using the following image processing steps: (i) a binary image was created from the raw image; (ii) a threshold level was set (the same level for all sections); (iii) ImageJ ‘Measure’ function was used to determine the percentage area of CD34 staining. Three random areas from the cortex were analysed for five animals from each group (normal MSC treatment, VEGF knockdown MSC treatment, and control vehicle treatment). Each random area included 10 high power fields that were analysed in the described stepwise, standardized fashion.

Statistical Analyses

Data are presented as means±S.D., unless otherwise stated. Statistical analyses were performed with GraphPad Prism 4 for Macintosh (GraphPad Software, San Diego, Calif., USA). ANOVA, t-test and Kaplan-Meier analysis were used to assess differences between data means as appropriate. All groups consisted of at least six animals. A P-value of <0.05 was considered significant.

Results

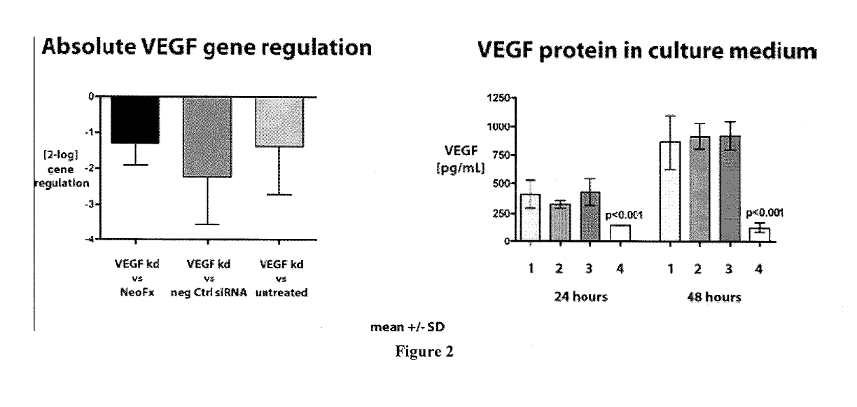

VEGF knockdown efficiency with siRNA Adherent MSCs in culture flasks were treated for 24 hrs with VEGF siRNA and NeoFX transfection agent in regular growth medium in order to knockdown VEGF expression. Knockdown efficiency was determined at RNA and protein levels 24 hrs and 48 hrs after the end of the transfection period. Efficiency of the knockdown approach was tested before each experiment and adjusted if necessary (combination of siRNAs or different concentrations). Initially, combination of three VEGF siRNA yielded a greater than 80% knockdown of VEGF at the RNA and protein levels. In later experiments, 10 nm of siRNA ID #192613 was used and yielded a greater than 60% knockdown at the protein level. Results are shown in FIG. 2. Knockdown was verified at the mRNA as well as the protein level.

In Vitro Studies with MSC Conditioned Medium

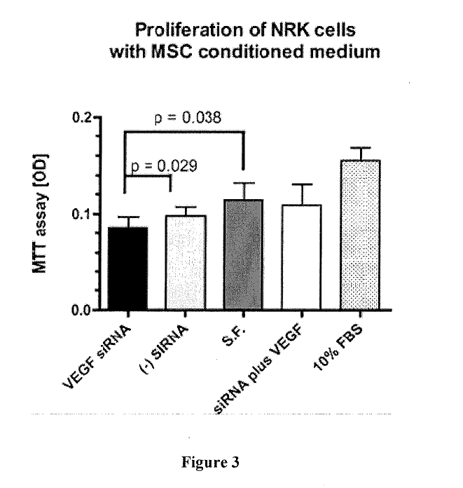

NRK cells express VEGF receptors Flt-1, flt-4 and flk-1 as determined by PCR and showed proliferative activity when VEGF was added to the medium (data not shown). VEGF knockdown with siRNA reduced VEGF protein levels in MSC conditioned medium (FIG. 2, right panel). Conditioned medium from VEGF knockdown MSC exerted less proliferative activity on proximal tubular cells compared to regular conditioned medium from MSCs treated with irrelevant siRNAs (P=0.029) or control medium (S.F. [serumfree medium], P=0.038) (FIG. 3), thereby demonstrating the mitogenic activity of VEGF in tubular cells. Addition of 10 ng/ml VEGF restored proliferative activity of the conditioned medium.

VEGF Knockdown Reduces Renoprotection of MSCs

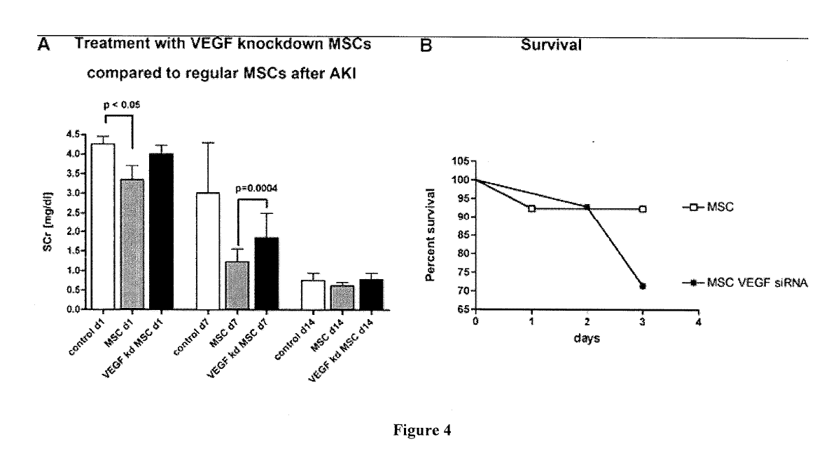

In order to investigate the applicability of our in vitro results to the in vivo situation, we studied the comparative renoprotective effects of wild type MSCs and to VEGF knockdown MSCs in the standard model of ischemia/reperfusion AKI. Female SD rats were subjected to 48 min. of bilateral renal pedicle clamping to induce severe AKI. Regular MSCs were renoprotective as shown by lower serum creatinine values on days 1 and 7 compared to vehicle injection (FIG. 4A). VEGF knockdown rendered MSCs less effective in exerting renoprotection and recovery, which was highly significant at day 7 (P=0.0004). Survival of animals treated with VEGF knockdown MSCs during the first three days after clamping was lower compared to MSC treated animals (FIG. 4B).

Decreased Microvessel Density After Treatment with VEGF Knockdown MSCs

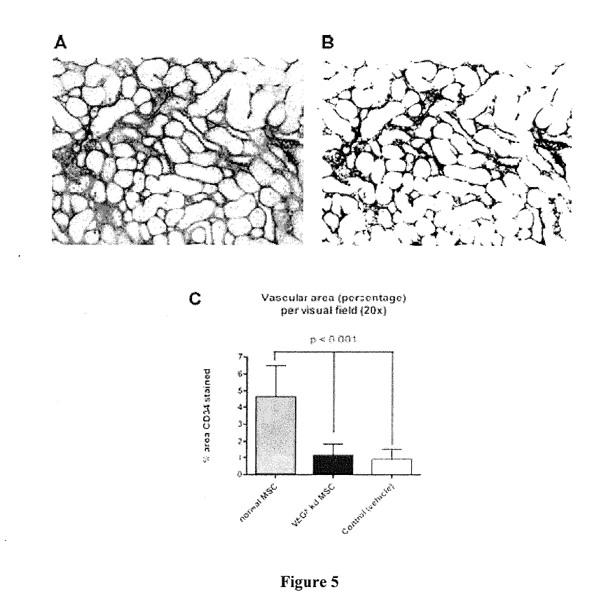

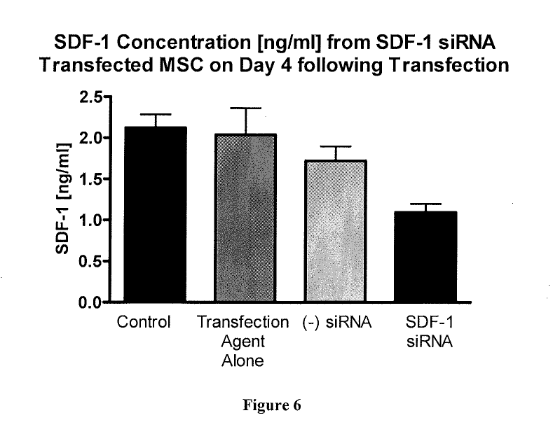

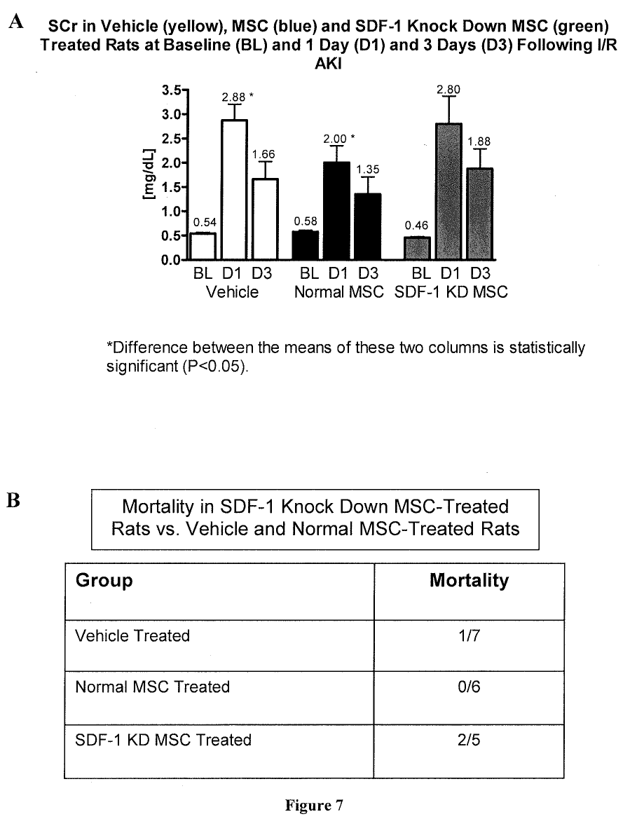

VEGF is the major mediator of vascular growth and repair and microvascular injury is an important pathophysiological component of AKI (Molitoris B A, et al. Kidney Int. 2004; 66: 496-9; Sutton T A, et al. Am J Physiol Renal Physiol. 2003; 285:F191-8). Therefore, we determined renal microvessel density at 4 weeks after ischemia/reperfusion AKI in animals treated with regular MSCs and VEGF knockdown MSCs, using an immunostaining approach. Paraffin sections of kidneys were immunostained with CD34 to visualize the renal vasculature (FIG. 5A). No counterstaining was applied and all sections were examined the same way with ImageJ. Area percentage of staining from binary images (FIG. 5B) was determined with the ‘measure’ function. Animals treated with regular MSCs had a significantly higher percentage area of vasculature compared to animals treated with VEGF knockdown MSCs and vehicle treated animals (FIG. 5C; P=0.001). Tissue injury (apoptosis/necrosis) was not determined at this time point since surviving animals had recovered from the acute phase of AKI and these data would not have added additional information.

Discussion

MSCs are bone marrow derived stem cells and, together with haematopoietic stem cells, are already in clinical use to treat patients with various diseases. Their effectiveness has been shown in a number of diseases, but the mechanism of action is incompletely defined and likely includes a multitude of actions, e.g. paracrine growth factor secretion, immunomodulation and anti-apoptotic properties. Since AKI is caused by multi-factorial pathophysiological mechanisms, including inflammation and vascular injury, MSCs appear to be suitable candidates for a cell based therapy of this common disease that is associated with high hospital mortality.

### Example 5

### Cryospreservation Protocol for Human Mesenchymal Stromal Cells (hMSCs)

Mesenchymal stromal cells were cryopreserved in a DMSO solution, at a final concentration of 10%, for long-term storage in vapor phase liquid nitrogen (LN2, <−150° C.). The viability and functionality of hMSCs in prolonged storage has been demonstrated and there is currently no recognized expiration of products that remain in continuous LN2 storage.

hMSCs were derived from human bone marrow.

Reagents, Standards, Media, and Special Supplies Required:

| | |
|---|---|
| Dimethyl Sulfoxide (DMSO) | Protide Pharmaceuticals |
| Human Serum Albumin 25% | NDC 0053-7680-32 |
| Plasmalyte A | |
| Cryovials | |
| Dispensing Pin | |
| 20 cc Syringe without Needle | |
| 30 cc Syringe without Needle | |
| 18 gauge Blunt Fill Needle | |
| Alcohol Preps | |
| Betadine Preps | |
| Ice Bucket | |
| 10 ml serological pipette | |
| 25 ml serological pipette | |
| 250 ml Conical Tube | |
| Cryogloves | |

Instrumentation:
Pipettes
Biological Safety Cabinet (BSC)
Controlled Rate Freezer (CRF)
LN2 Storage Freezer with Inventory System
Centrifuge

A. Calculate the Number of Cyrovials Needed to Freeze the hMSC Product
1. Calculating Freeze Mix: The number of cryovials necessary to freeze a give quantity of cells was calculated. The cells are stored at 15×10⁶/ml. Thus, the number of cells present was divided by this number to ascertain the volume of cells and medium to be frozen.

For example, 3.71 ×10⁸=24.7 ml.
2. Calculating number of cryovials: The number of vials needed for a given volume of cells plus medium was calculated. The volume of the cryovials was 1 ml or 4m1. Thus, the volume calculated above was divided into the number of cryovials needed.

For example: 24 ml=6, 4 ml cyrovials

B. Calculate the Total Freeze Volume

Total freeze volume consisted of 10% DMSO by volume, 20% albumin by volume, and the remaining volume Plasmalyte (70%).
For example: Total Freeze Volume=24 ml
   DMSO=2.4 ml
   Albumin=4.8 ml
   Plasmalyte=16.8 ml

C. Prepare Freeze Mix
1. Ice bucket prepared.
2. The desired volume of DMSO was obtained with an appropriate sized syringe.
3. The same volume of plasmalyte that was obtained.

a. e.g. 6 ml of DMSO, 6 ml of plasmalyte
4. The DMSO and plasmalyte were added to the “Freeze Mix” tube.
5. The solution was mixed and placed on ice to chill for at least 10 minutes.
6. The albumin was placed on ice

D. Prepare Sample for Freezing
1. The final product was centrifuged in a 250 ml conical tube at 600×g (−1600 rpm) for 5 minutes, no brake.
2. The supernatant was removed to one inch above the cell pellet using a 25 ml serological pipette, The cell pellet was not disturbed.
3. The supernatant was removed and placed in a sterile 250 ml conical tube labeled “Sup”.
4. Both the cells and supernatant were placed on ice

E. Freezing
1. The amount of plasmalyte still needed for the freeze mix was calculated and the desired volume was obtained.

a. For example, the volume of DMSO +the volume of already added plasmalyte+the volume of albumin+cell pellet volume minus the total freeze volume equals amount of plasmalyte needed.
2. The albumin bag was aseptically spiked with a dispensing pin and the desired volume of albumin was removed.
3. The albumin and plasmalyte were added to the “Freeze Mix” tube and mixed.
4. Using a 10 ml serological pipette the chilled freeze mix aseptically removed and added slowly to the resuspended cells. While adding the freeze mix cells were gently mixed by swirling. Once the Freeze Mix was added to the product, the freeze was initiated within 15 minutes. If a delay was expected, the product mixture was placed back on ice. Under no circumstances was the mix allowed to be unfrozen for more than 30 minutes.
5. The lid was placed on the tube containing cell mix and the tube was inverted several times to mix the contents.
6. Using a 10 ml serological pipette the freeze volume was aseptically removed and the appropriate volume was dispensed into each labeled cryovial. In 1.8 ml vials Iml of cell mix was placed. In 4.5 ml vials 4 ml of cell mix was placed.
7. The cryovials were then immediately placed on ice and then frozen using the controlled rate freezer to −80° C.

F. Expected Ranges for MSCs Thawed After Being Frozen According to Protocol:
1. Thawed Product Viability >70%
2. Sterility Testing=Negative
3. Differentiation=growth for adipogenic, osteogenic, and chondrogenic
4. Flow cytometry

a. CD 105 (≧90%)

b. CD 73 (≧90%)

c. CD 90 (≧90%)

d. CD 34 (<10%)

e. CD 45 (<10%)

f. HLA-DR (<10%)
5. Endotoxin <5.0 EU/kg body weight
6. Mycoplasma=negative

### Example 6

### Thawing Protocol for Human Mesenchymal Stromal Cells (hMSCs)

Stored human Mesenchymal stromal cells (hMSC) are cryopreserved using DMSO as a cell cryoprotectant. When thawed, DMSO creates a hypertonic environment which leads to sudden fluid shifts and cell death. To limit this effect, the product was washed with a hypertonic solution ameliorating DMSO's unfavorable effects. Post-thaw product release testing was done to ensure processing was performed so as to prevent contamination or cross-contamination.

Reagents, Standards, Media, and Special Supplies Required.

| | |
|---|---|
| Human Serum Albumin (HSA) 25% | NDC 52769-451-05 |
| Plasmalyte A | |
| Trypan Blue | |
| 300 ml Transfer Pack | |
| 15 ml conical tube | |
| 50 ml conical tube | |
| 250 ml Conical Tube | |
| 150 ml Transfer Pack | |
| Sterile Transfer Pipette | |
| 1.5 Eppendorf tube | |
| Red Top Vacutainer Tubes or equivalent | |
| 10 cc syringe | |
| 20 cc syringe | |
| 30 cc syringe | |
| 60 cc syringe | |
| 5 ml serological pipette | |
| 10 ml serological pipette | |
| Ice Bucket | |
| Blunt End Needle | |
| 200-1000 μl sterile tips | |
| Cryogloves | |
| Biohazard Bag | |
| Iodine | |
| Alcohol wipes | |

Instrumentation:
Biological Safety Cabinet (BSC)
Centrifuge
Sterile Connecting Device
Microscope, Light
Thermometer
Water Bath
Hemacytometer
Pipettes
Computer with Freezerworks
Ambient Shipper

A. Wash Solution Preparation
1. The cell dose required for infusion was calculated based on the recipient's weight. The required number of cells for infusion based on recipient weight was calculated by multiplying the cell dosage per kg times the recipient weight in kg to arrive at the number of cells necessary.
2. The number of cryovials needed to achieve the calculated cell dose was then determined. a. 1 ml of cell mix contains 15×10⁶ cells.
3. The wash solution volume needed to thaw all required cryovials was then calculated: For the example below, all numbers listed below are for a 100 kg patient.

a. Volume of product, multiplied times 4 in addition to 80 mls for cell resuspension and testing
1) for a dose of 7×10⁵ cells=˜7 mls of product thawed and a wash solution volume of 108 ml was used;
2) for a dose of 2×10⁶ cells=˜19 mls of product thawed and a wash solution volume of 156 ml was used;
3) for a dose of 5×10⁶ cells=˜46 mls of product thawed and a wash solution volume of 264 ml was used.

b. Wash Solution=20% by volume stock albumin (25% Human, USP, 12.5 g/50 ml), 80% Plasmalyte
4. A female end was sterile connected to a 300 ml transfer pack.
5. Using sterile technique, a calculated volume of Plasmalyte was removed and placed in a transfer pack.
6. The calculated volume of albumin was removed and the volume added to the Plasmalyte.
7. The bag was mixed well, placed in a tube on ice and solution was allowed to chill for at least 10 minutes

B. Thawing and Washing
1. The exterior of the cryovial containing the hMSCs was wiped with 70% alcohol and placed in a bucket with ice.
2. Each vial was thawed one at a time
3. The vial was wiped down with 70% alcohol and place in the biological safety cabinet.
4. Using a 5 ml serological pipette thawed product was removed and place in the labeled “Thaw and Washed Product ” tube.
5. Using an appropriate sized serological pipette the required amount of wash solution was removed (vial volume times 4).

a. The wash solution was slowly added drop wise to the thawed product. The was solution was gradually introduced to the cells while gently rinsing the product to allow the cells to adjust to normal osmotic conditions. Slow addition of wash solution with gentle agitation prevents cell membrane rupture from osmotic shock during thaw.

b. 1 ml of the wash solution was used to rinse the cryovial.

c. The rinse was added to the product conical tube.
6. The conical tube was placed on ice and retrieve the next vial
7. Steps 1-5 were repeated for any remaining vials.

a. For higher doses the volume was split in half, with one half of the volume thawed in one 250 ml conical tube and the other half in the other 250 ml conical tube.
8. The Thaw and Washed Product tube was centrifuged at 500 g for 5 min. with the brake on slow.
9. A serological pipette was used to slowly remove the supernatant (approximately one inch from the cell pellet)
10. The cell pellet was resuspended in 5 ml of wash solution.

a. For higher doses
1) The cell pellets were resuspended in the remaining supernatant
2) The cell pellets were combined.
3) 5 ml of wash solution was used to rinse the conical tube in which the cell pellet was removed and add wash solution to the product.

### REFERENCES

Lange, C., et al., Accelerated and safe expansion of human mesenchymal stem cells in animal serum-free medium for transplantation and regenerative medicine. J. Cell. Physiol. 213:18-26, 2007.

Togel, F. et al., VEGF is a mediator of the renoprotective effects of multipotent marrow stromal cells in acute kidney injury. J. Cell Mol. Med. 13:1-6, 2009.

Gooch, A., et al., Knock Down of Stromal Derived Factor-1 in Mesenchymal Stem Cells significantly impairs their protective Action in Rats with Acute Kidney Injury. Am. Soc. Neph.

1. A method of assaying the therapeutic effectiveness of mesenchymal stromal cells (MSCs) for treating a pathology in a subject comprising:
(a) isolating a first population of MSCs, wherein the first population of MSCs has been freshly isolated;
(b) isolating a second population of MSCs, wherein the second population has been passaged and/or frozen and thawed;
(c) measuring the expression of stromal derived factor-1 (SDF-1) and/or vascular endothelial growth factor (VEGF) in the first and second populations; and
(d) comparing the expression of SDF-1 and/or VEGF in the first and second populations;
wherein, if the expression of SDF-1 and/or VEGF in the second population is the same as or greater than the expression of SDF-1 and/or VEGF in the first population the second population contains MSCs that are therapeutically effective.

2. The method of claim 1, wherein the MSCs from the first and second populations are autologous to the subject.

3. The method of claim 2, wherein the subject is a mammal.

4. The method of claim 3, wherein the mammal is a human.

5. The method of claim 1, wherein the MSCs from the first and second populations are allogeneic to the subject.

6. The method of claim 5, wherein the subject is a mammal.

7. The method of claim 6, wherein the mammal is a human.

8. The method of claim 1, wherein the MSCs from the first and second populations are isolated at different times.

9. The method of claim 1, wherein the time between the isolation of the first and second populations is about 1 day apart.

10. The method of claim 9, wherein the time between the isolation of the first and second populations is about 1 week apart.

11. The method of claim 9, wherein the time between the isolation of the first and second populations is about 1 year apart.

12. The method of claim 9, wherein the time between the isolation of the first and second populations is greater than one year apart.

13. The method of claim 1, wherein the first and second populations are isolated at about the same time.

14. The method of claim 1, wherein the pathology is selected from the group consisting of a neurological pathology, an inflammatory pathology, a renal pathology, a hepatic pathology, a cardiovascular pathology, a retinal pathology, a muscular pathology, a bone-related pathology, a gastrointestinal pathology, a skin related pathology and a metabolic pathology.

15. The method of claim 14, wherein the renal pathology is selected from the group consisting of acute kidney injury, acute renal failure, chronic renal failure, chronic kidney disease and transplant.

16. The method of claim 14, wherein the neurological pathology is stroke.

17. The method of claim 14, wherein the inflammatory pathology is multi-organ failure.

18. The method of claim 14, wherein the metabolic pathology is diabetes.

19. A method of treating an MSC related pathology in a subject in need thereof comprising:
(a) isolating a first population of MSCs, wherein the first population of MSCs has been freshly isolated;
(b) isolating a second population of MSCs, wherein the second population has been passaged one or more times and/or frozen and thawed;
(c) measuring the expression and/or secretion into the media of stromal derived factor-1 (SDF-1) and/or vascular endothelial growth factor (VEGF) in the first and second populations; and
(d) comparing the expression of SDF-1 and/or VEGF in the first and second populations;
wherein, if the expression of SDF-1 and/or VEGF in the second population is the same as or greater than the expression of SDF-1 and/or VEGF in the first population the second population contains MSCs that are therapeutically effective; and a therapeutically effective dose of the MSCs in the second population is administered to the subject, thereby treating the MSC related pathology in the subject.

20-36. (canceled)

37. A kit comprising reagents for the detection of the expression of SDF-1 and reagents for the detection VEGF.

38-41. (canceled)

42. A method of producing a dosage form of MSCs comprising:
(a) isolating a first population of MSCs, wherein the first population of MSCs has been freshly isolated;
(b) isolating a second population of MSCs, wherein the second population has been passaged one or more times and/or frozen and thawed;
(c) measuring the expression of stromal derived factor-1 (SDF-1) and/or vascular endothelial growth factor (VEGF) in the first and second populations; and
(d) comparing the expression of SDF-1 and/or VEGF in the first and second populations;
wherein, if the expression of SDF-1 and/or VEGF in the second population is the same as or greater than the expression of SDF-1 and/or VEGF in the first population the second population of MSCs are combined with a physiologically acceptable solution, thereby producing a dosage form of MSCs.

43-54. (canceled)
